(19) United States
(12) Patent Application Publication
(10) Pub. No.: US 2021/0085257 A1
(43) Pub. Date: Mar. 25, 2021

(54) SYSTEM AND METHOD TO PREDICT TARGET VOLUME REGION FOR THERAPEUTIC TISSUE ACTIVATION

(71) Applicant: THE REGENTS OF THE UNIVERSITY OF MICHIGAN, Ann Arbor, MI (US)

(72) Inventors: Parag G. Patil, Ann Arbor, MI (US); Charles W. LU, Ann Arbor, MI (US)

(21) Appl. No.: 16/582,734

(22) Filed: Sep. 25, 2019

(51) Int. Cl. A61B 5/00; A61B 5/0478; A61N 1/05

(52) U.S. Cl. CPC A61B 5/7275; A61N 1/0534; A61B 5/7264; A61B 5/0478

(57) ABSTRACT

A neural targeting system and method for determining placement of a stimulation probe within the brain for stimulation treatment of an individual afflicted with an illness or disorder. Electrophysiological data attained within the brain of the individual is utilized with clinically-determined stimulation treatment of a plurality of similarly-afflicted individuals to predict a tissue activation volume within the brain of the individual for stimulation treatment.

### FIELD OF TECHNOLOGY

The following disclosure relates to therapeutic tissue stimulation for the treatment of an illness or disorder, and more particularly, to using machine learning techniques to predict a volume tissue within the brain for stimulation.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Deep brain stimulation (DBS) therapy is a known treatment for various human illnesses and disorders, such as essential tremor, dystonia, epilepsy, and Parkinson disease. The effectiveness of the treatment DBS treatment relies on a time-consuming empirical examination by medical personnel, e.g., neurosurgeon, neurologist; to determine an optimal site within an individual's brain for tissue activation. Final determination of the stimulation site involves trial-and-error attempts by the neurologist, which typically requires multiple sessions spanning several months. The tissue activation site may vary significantly across individuals and is typically analyzed as a solitary point, although improved therapeutic tissue stimulation may likely be achieved through stimulation of multiple distinct neural populations, e.g., volume, of brain tissue for activation. Therefore, a clear and unmet need exists to improve the efficiency of therapeutic tissue stimulation for an individual and to more accurately predict a three-dimensional span for therapeutic stimulation.

### SUMMARY

Described herein are example systems and methods for predicting a volume of brain tissue for stimulation or activation, which may facilitate a more effective therapeutic treatment for a variety of neurological-related illnesses and disorders. In general, machine-learning techniques are utilized to map high-density electrophysiology to clinically-validated areas of brain tissue and surveyed for predicative electrophysiological markers. For example, a machine learning model is trained on electrophysiology data deduced from recorded electrophysiological signals received via a microelectrode traversing one or more trajectories within the brain of a plurality of individuals afflicted with a neurological-related illness or disorder along with the resulting clinically-determined stimulation parameters employed to effectively treat each individual. The trained machine learning model is capable of utilizing electrophysiology data attained via a microelectrode traversing one or more trajectories within the brain of a similarly-afflicted individual and then predict the tissue activation volume (VTA), e.g., stimulation parameters, for targeted stimulation treatment of the individual.

Accordingly, one example method for predicting a location within the brain of an individual afflicted with an illness or disorder for stimulation treatment includes: extending a microelectrode along a trajectory within the brain of the individual; receiving, via the microelectrode, electrophysiology data at a plurality of intervals along the trajectory, the electrophysiology data being indicative of neural activity of the individual; and, utilizing, via one or more processors, a machine learning model trained on clinically-determined stimulation treatment of a plurality of similarly-afflicted individuals to predict a tissue activation volume within the brain of the individual based on the electrophysiology data received along the trajectory.

Another example method is directed to adjusting the stimulation treatment of an initial volume within the brain of an individual afflicted with an illness or disorder. The method includes receiving electrophysiology data via a stimulation probe implanted with the brain of the individual, the implanted stimulation probe configured to stimulate the initial volume within the brain; utilizing, via one or more processors, a machine learning model trained on clinically-determined stimulation treatment of a plurality of similarly-afflicted individuals to predict the tissue activation volume with the brain of the individual based on the received electrophysiology data of the individual; analyzing the predicted tissue activation volume within the brain with the stimulated initial volume within the brain; and adjusting stimulation parameters of the stimulation probe based on the analysis of the predicted tissue activation volume with the stimulated initial volume to stimulate the predicted tissue activation volume within the brain of the individual.

Another example method for predicting a location within the brain of an individual afflicted with an illness or disorder for stimulation treatment includes: extending a microelectrode along a plurality of trajectories within the brain of the individual; receiving, via the microelectrode, electrophysiology data at a plurality of intervals along each of the plurality of trajectories; the electrophysiology data being indicative of neural activity of the individual; utilizing, via one or more processors, a machine learning model trained on clinically-determined stimulation treatment of a plurality of similarly-afflicted individuals to predict a plurality of tissue activation volumes with the brain of the individual based on the electrophysiology data received along the plurality of trajectories, wherein each of the predicted plurality of tissue activation volumes associated with a respective one of the plurality of trajectories; analyzing the predicted tissue activation volumes based on a stimulation treatment criteria to determine a tissue activation volume for stimulation treatment; and, selecting one of the plurality of trajectories associated with the determined tissue activation volume for treatment of the individual.

A further example embodiment for predicting a location within the brain of an individual afflicted with an illness or disorder for stimulation treatment includes training a machine learning model using clinically-determined stimulation treatment of a plurality of similarly-afflicted individuals. The clinically-determined stimulation treatment may include electrophysiology data received via a microelectrode probe inserted along one or more trajectories within the brain of one or more similarly-afflicted individuals; one or more clinically-determined tissue activation areas within the brain of the one or more of the similarly-afflicted individuals; and/or the corresponding stimulation parameters of the clinically-determined tissue activation areas within the brain of the one or more similarly-afflicted individuals.

Yet another example embodiment for predicting a location within the brain of an individual afflicted with an illness or disorder for stimulation treatment includes a system comprising one or more processors coupled to a microelectrode receiving electrophysiology data along one or more trajectories within the brain of the individual. The one or more processors are also coupled to a non-transitory computer-readable memory storing instructions thereon, which when executed by the one or more processors, cause the system to perform any of methods described herein. Additionally, any of the methods herein described may include providing the predicted tissue activation volume and corresponding stimulation parameters for presentation to stimulation treatment personnel.

### BRIEF DESCRIPTION OF THE DRAWINGS

The figures described below depict various aspects of the systems and methods disclosed therein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 illustrates a flow diagram of an example method for predicting a tissue activation volume within the brain of an individual afflicted with an illness or disorder and performed in accordance with the embodiments described herein.

FIG. 2 illustrates two sets of electrophysiology data of treated individuals, namely a training set and a test set (depicted within a dash-lined box).

FIG. 3 is an enlarged image of one illustration of the electrophysiology data of an individual within the training set shown in the upper row and furthest left of FIG. 2.

FIG. 4 is a graph of a receiver operating characteristic curve of a classifier calculated from the test data depicted within the dash-lined box of FIG. 2.

FIG. 5 is a table illustrating classifier performance of the full model and the beta-only model.

FIG. 6 illustrates a flow diagram of another example method for predicting a tissue activation volume within the brain of an individual afflicted with an illness or disorder and performed in accordance with the embodiments described herein.

FIG. 7 illustrates an exemplary block diagram of an embodiment of a neural targeting system assembled in accordance with the teachings of the present invention and used to predict a tissue activation volume within the brain of an individual for therapeutic stimulation.

### DETAILED DESCRIPTION

Described herein are example systems and methods for predicting a volume of brain tissue for therapeutic stimulation, e.g., activation, which may be used to clinically treat individuals afflicted with neurologically-related illnesses or disorders, e.g., brain tumors, epilepsy, hydrocephalus, movement disorders, etc. In general, machine learning techniques, e.g., machine learning model, support vector machine, etc., are utilized to map high-density, broadband electrophysiology to a clinically-validated volume of brain tissue for therapeutic stimulation. For example, a machine-learning model (trained with electrophysiology data attained from a plurality of other similarly-afflicted and therapeutically treated individuals) is utilized in conjunction with electrophysiology data of an individual to be treated for predicting a tissue activation volume (VTA) for the individual. By incorporating electrophysiology of the individual with tissue activation volumes modeled from clinically-determined therapeutic stimulation characteristics of other similarly-afflicted individuals, a more accurate tissue activation volume within the brain of the individual may be predicted in significantly less time, e.g., significantly fewer office sessions, than typically accomplished.

To more precisely predict the three-dimensional span of brain tissue to be activated by therapeutic stimulation, machine learning techniques may be used to analyze individualized anisotropic models of therapeutic tissue activation along with high-density broadband electrophysiology, which is then used to map recorded electrophysiology data to clinically-derived tissue activation models. For example, a machine learning model is generated from machine learning techniques, such as a regression analysis (e.g., a logistic regression, linear regression, or polynomial regression), k-nearest neighbors, decisions trees, random forests, boosting, neural networks, support vector machines, deep learning, reinforcement learning, Bayesian networks, etc. One example machine learning model utilized to predict a tissue activation volume for therapeutic treatment of an individual, e.g., prediction model, is based on clinically-derived tissue activation treatment of similarly-afflicted individuals in conjunction with electrophysiology data of the individual to be treated. This data-driven approach identifies associations between regions of therapeutic tissue activation and broadband electrophysiological features, including cross-frequency interactions. By incorporating an individual's heterogeneous anisotropy into the machine learning model, a more precise individual-specific estimate of tissue activation volume may be attained.

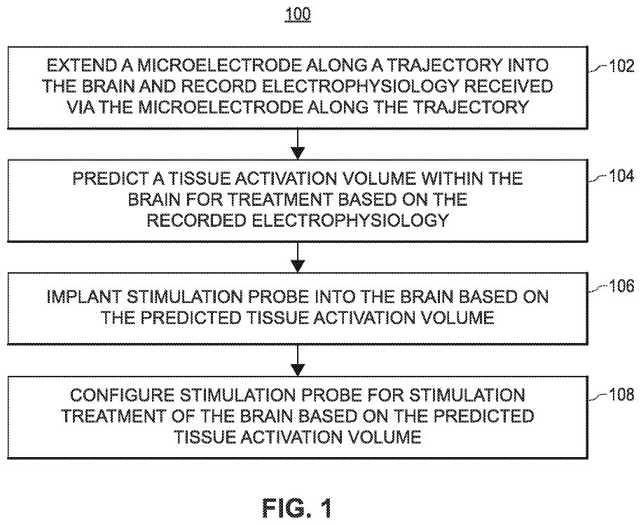

A flow diagram of one implementation of an example method 100 for predicting a volume within the brain for stimulation treatment, e.g., deep brain stimulation (DBS), of an individual afflicted with an illness and/or disorder is shown in FIG. 1. Initially, a data structure may be constructed of stimulation treatment data of a plurality of other similarly-afflicted individuals. Composition of the data structure may include intraoperative electrophysiology data, e.g., neurological signals, stimulation parameters, etc., of the plurality of other treated individuals, which may be deduced from recorded electrophysiology activity received via a microelectrode traversing one or more trajectories within the brain of the plurality of individuals, and mapped into a respective machine learning model by direct visualization of the stimulation probe on a computed tomography (CT). Similarly, to attain electrophysiology data of the individual to be treated, a microelectrode is inserted into the brain along one or more trajectories. Generally, the electrophysiology data includes normal neural activity of the individual, however it may also include neural activity in response to one or more stimuli, e.g., electrical, chemical, mechanical, and/or other sensory prompt, administered to the individual. Any attained electrophysiology data may be utilized with a machine learning model to predict the volume of brain tissue for activation, e.g., stimulation treatment (block 104). The predicted tissue activation volume may then be utilized by treatment personnel to treat the afflicted individual. For example, treatment personnel may implant a stimulation probe proximate the predicted tissue activation volume (block 106), and configure the stimulation probe accordingly (block 108).

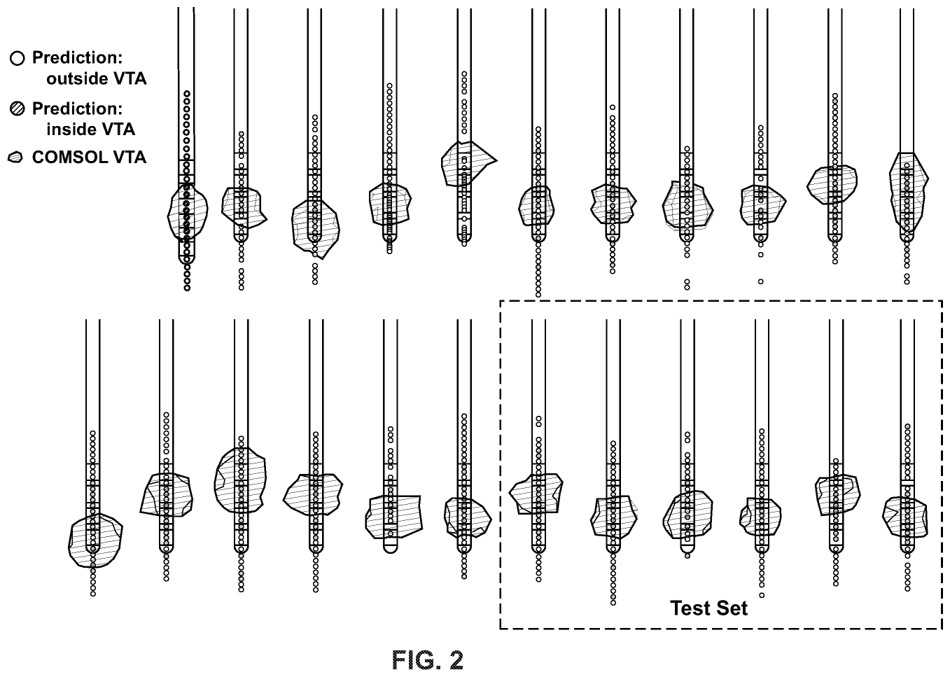

FIG. 2 depicts illustrations of two sets of recorded electrophysiology data, any of which may be stored and organized in a data structure and accessible to one or more processors and machine learning models for implementing in a therapeutic treatment system. A first set, e.g., training set, of recorded electrophysiology data (illustrated outside of the dashed-box) is training data used to generate a machine learning model, e.g., prediction model, incorporated with the system for predicting a volume (e.g., tissue activation volume) within the brain for stimulation treatment of an individual afflicted with a neurological-related illness or disorder. The electrophysiology data of the training set is attained via one or more microelectrodes inserted along one or more trajectories within the brain of other similarly-afflicted individuals. A stimulation probe or lead for providing stimulation treatment (e.g., deep brain stimulation (DBS) of the subthalamic nucleus (STN)) was permanently implanted within one of the trajectories within the brain of each individual. Intraoperative fluoroscopy was used to confirm alignment of the corresponding permanently implanted stimulation probe with the trajectory of each microelectrode recording.

The electrophysiology of each individual in the training set was spatially mapped to a stimulation area along the trajectory, which was generated in COMSOL Multiphysics® with respect to each individual's clinical stimulation parameters, e.g., MRI scan, stimulation probe, amplitude. The training set of data includes electrophysiology recorded using the microelectrode, for example, at 0.5 mm intervals along each trajectory and between approximately 15 mm above and 5 mm below a desired target. At each interval (i.e., depth), neurological-related activity, e.g., spike rate, log of normalized power; was calculated with respect to one or more stimulation criteria, for example, various frequency bands and/or ranges, e.g., delta (0.1-4 Hz), theta (4-8 Hz), alpha (8-13 Hz), beta (13-30 Hz), low gamma (30-59 Hz), high gamma (61-200 Hz), high frequency oscillation (HFO; 200-400 Hz) and high frequency band (HFB; 500-2000 Hz). In addition to such main effects, first order interaction terms may also be calculated, e.g., log(beta power) x log(low gamma power).

A machine learning model, e.g., prediction model, was trained on the training set of data, wherein each recorded interval of electrophysiology data was analyzed with respect to the clinically-determined tissue activation volume. Machine learning techniques, for example, statistical analysis, was applied to the electrophysiology data of the training set to identify predictors associated with the clinically-validated therapeutic stimulation areas. A regression method such as logistic LASSO (least absolute shrinkage and selection operator) may be utilized to identify electrophysiology parameters, aspects, or features that provide a predictive value(s) for identifying tissue activation volumes. Several neural features that were identified as potentially providing a predictive value include: high frequency band (HFB), theta×HFB, alpha×beta, beta×HFB, and high gamma×high frequency oscillation (HFO). Additionally, a classifier, e.g., support vector machine, analyzed each recorded interval of electrophysiology data, which is shown superimposed with the stimulation probe and its trajectory and classified as being located inside or outside the area of therapeutic stimulation.

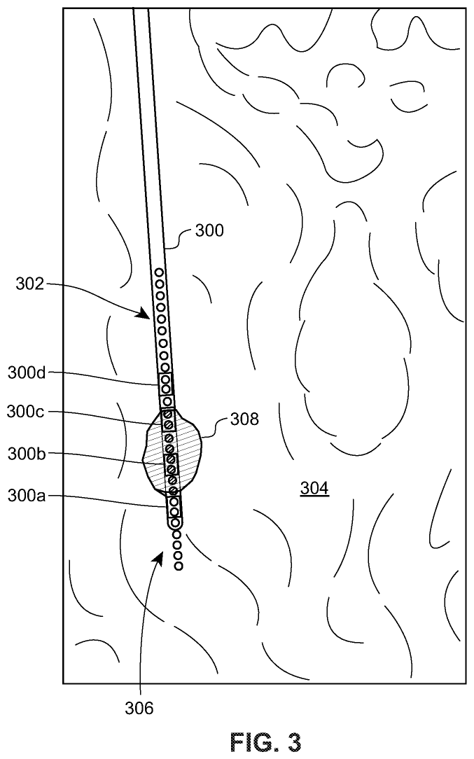

FIG. 3 is an enlarged image of one illustration of the electrophysiology data of an individual within the training set shown in the upper row and furthest left of FIG. 2, wherein the permanently implanted stimulation probe 300, its trajectory 302 within the brain 304, and the clinically-determined activation area 308 are shown in conjunction with the recorded intervals 306 of electrophysiology data attained separately with a microelectrode. Each interval 306 of sensed electrophysiology along the trajectory is classified to be inside or outside of the predicted tissue activation volume. For example, the machine learning model was trained on the training set of data with an assumption that the empirical prior probabilities of an interval being inside or outside the clinically-determined activation area 308 were uniform. Intervals 306 within the trajectory 302 that were predicted to reside within the clinically-determined activation area 308 are depicted as an encircled slash or diagonally-slanted line (“/”), and interval locations 306 within the trajectory 302 that were predicted to reside outside the clinically-determined activation area 308 are depicted as an empty circle.

Performance of the prediction model to classify intervals of recorded electrophysiology data (i.e., inside or outside a predicted tissue activation volume) was determined using a second set, e.g., test set, of recorded electrophysiology data (illustrated inside of the dashed-box in FIG. 2). The prediction model utilized a support vector machine classifier with one or more electrophysiological features (e.g., alpha, beta, theta, high gamma, HFO, HFB, theta×HFB, alpha×beta, beta×HFB, high gamma×HFO) suggested by logistic regression, i.e., LASSO, to predict therapeutic tissue activation sites, i.e., tissue activation volume. The prediction model achieved 64% sensitivity and 82% specificity on the test set of independently analyzed intervals and outperformed a beta-only classifier, and a simple anatomical approach to targeting and programming. See FIG. 5. These results are a significant improvement in performance over beta-only classifications, which is commonly used by treatment personnel.

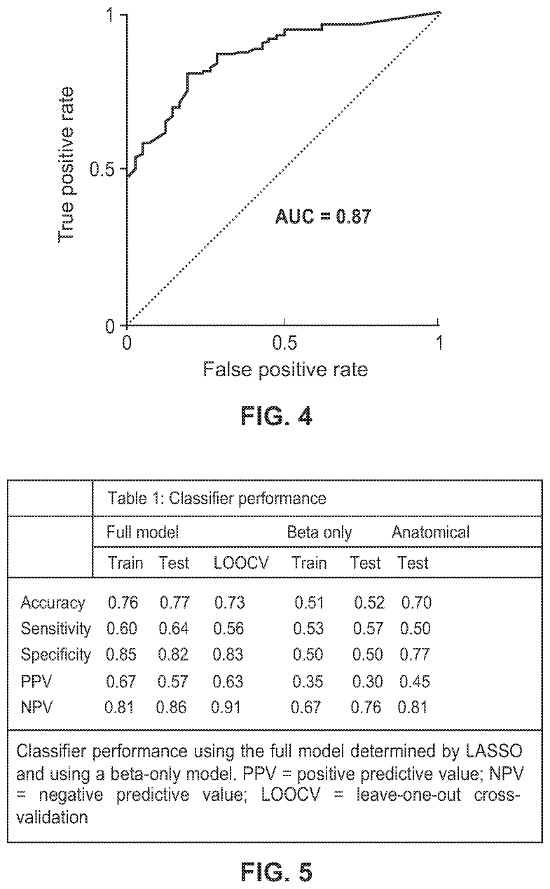

Additionally, while conventional therapeutic activation sites are generally discrete points, the prediction model yielded observations that a more clinically effective therapeutic stimulation treatment may be attained by stimulating a volume within the brain, which may include, span, or be associated with several intervals, i.e., contiguous or non-contiguous; as well as stimulating multiple volumes (e.g., non-contiguous) within the brain. To this end, statistically smoothing the classified intervals of electrophysiology data along the trajectory yielded probabilistic scores indicative of a higher accuracy and generalizability across individuals, as illustrated in FIG. 4 by the area under the curve (AUC) of 0.87 in the graph of true positive rates vs. false positive rates for the test data of FIG. 2.

To stimulate one or more tissue activation volumes within the brain, the stimulation probe 300 may include one or more excitation contacts 300a, 300b, 300c, 300d and/or one or more leads including one or more excitation contacts. Effective placement of the stimulation probe 300 may be based on the proximity of one or more of its excitation contacts 300a, 300b, 300c, 300d (and/or leads) to the predicted tissue activation volume(s) and any desired stimulation characteristic to be implemented (e.g., signatures, such as frequency, pulse, amplitude, phase, probe depth, etc.). For example, assuming now that the tissue activation area 308 illustrated in FIG. 3 is the predicted tissue activation volume of an individual to be treated, a more effective therapeutic stimulation may be attained by activation of the second and third excitation contacts 300b, 300c of the stimulation probe 300, which coincide with the classified interval locations 306 within the trajectory 302 that were calculated to reside within the predicted tissue activation volume. In contrast, the location of an activated contact of the stimulation probe that is not closely aligned with a one or more intervals of recorded electrophysiology that were classified to reside inside the predicted tissue activation volume may be indicative of a less efficient placement of the stimulation probe and its activated contact(s).

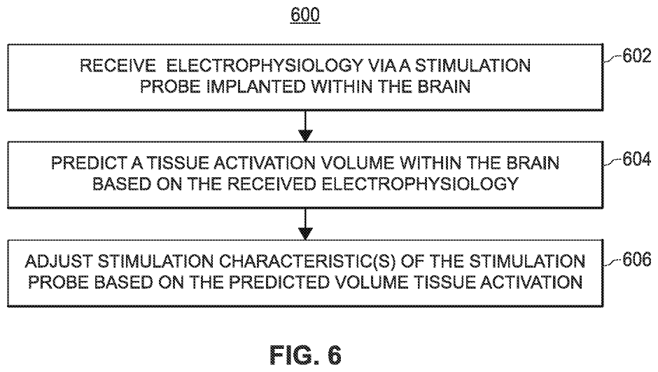

A flow diagram of an implementation of another example method (600) for predicting a tissue activation volume within the brain for stimulation treatment, e.g., deep brain stimulation (DBS), is depicted in FIG. 6. This method is directed to modifying the stimulation characteristics of a stimulation probe that has already been permanently implanted within the brain of an afflicted individual receiving neurological stimulation treatment. The method envisions the stimulation probe also having a sensing functionality and being capable of receiving electrophysiology data (block 602), albeit at a less-refined capability, e.g., resolution, than that capable of being received with a microelectrode probe (e.g., 0.5 mm intervals), and utilizing the prediction model to predict the tissue activation volume based on the electrophysiology data received by the stimulation probe (block 604). The predicted tissue activation volume is then analyzed, e.g., compared, to the initial, existing region of brain tissue being treated/targeted. Based upon the analysis of the predicted tissue activation volume and the initial or current region of brain tissue being stimulated, the stimulation parameters are adjusted to activate the permanently implanted stimulation probe (e.g., one or more excitation contacts) to stimulate the predicted tissue activation volume activation/stimulation treatment (block 606). Adjustments and/or modification to the stimulation parameters may include duration, amplitude, and/or frequency of the stimulation pulse train. In this way, the stimulation characteristics of the previously and permanently implanted stimulation probe may be modified to provide a more effective stimulation treatment to the individual by adjusting the initially prescribed stimulating characteristics without needing additional surgical procedures to remove/replace the stimulation probe and/or attain electrophysiological data via additional insertions of the microelectrode sensing probe.

An alternative technique for adjusting the stimulating parameters of the permanently implanted stimulation probe based on the individual's electrophysiology data that is received by the permanently implanted stimulation probe is to use the individual's recorded electrophysiology data that was previously acquired, for example, during initial probing of the microelectrode into the individual's brain, which was likely performed prior to, and in preparation of, permanently implanting the stimulation probe. As with the electrophysiology data attained via the permanently implanted stimulation probe, the individual's initially attained electrophysiology data may be utilized with the prediction model to predict the tissue activation volume for stimulation treatment, e.g., activation; wherein the predicted tissue activation volume may then be utilized by treatment personnel to treat the afflicted individual. For example, the initial and/or current tissue activation point and/or stimulation characteristics of the permanently implanted stimulation probe (e.g., frequency, amplitude, stimulation contact(s), etc.) may be analyzed and/or compared with the predicted tissue activation volume and/or corresponding stimulation characteristics, and based upon the analysis/comparison, the initial/current stimulation characteristics of the permanently implanted stimulation probe may be modified and/or adjusted to stimulate the predicted tissue activation volume. Without physically adjusting the location of the permanently implanted stimulation probe, any combination of its stimulation parameters, e.g., frequency[ies], amplitude(s), stimulation contact(s), etc. may be modified/adjusted to effectively stimulate the three-dimensional region within the brain that is more closely aligned with the predicted tissue activation volume.

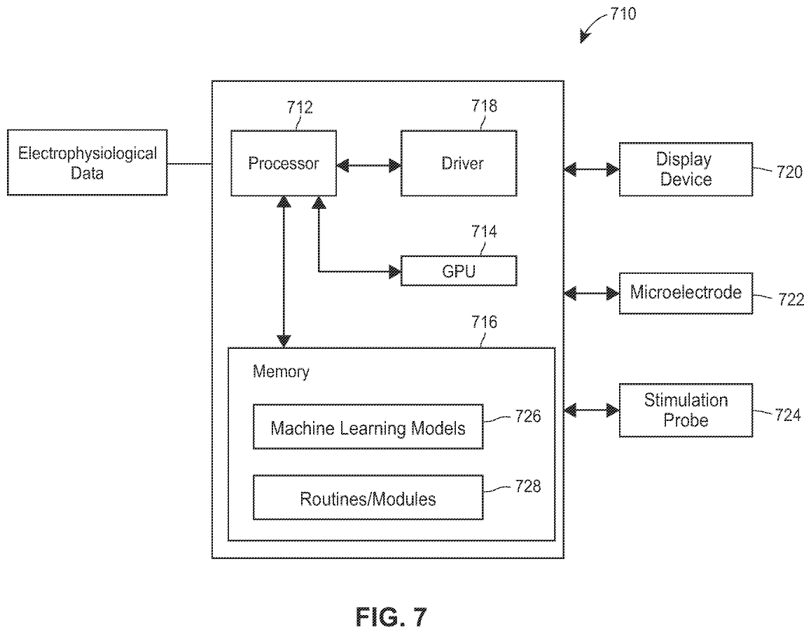

FIG. 7 is a block diagram of one embodiment of a neural targeting system 710 capable of executing the example methods described herein for predicting a tissue activation volume for stimulation treatment within the brain of an individual afflicted with an illness or disorder. The system 710 includes one or more processors 712, one of which may be a graphical processor 714, operatively coupled to a memory 716 and a user interface, i.e., display device 720, for presenting stimulation information, such as images of the predicted tissue activation volume, trajectories within the brain, stimulation probe, microelectrode, etc. In some configurations, the display device 720 is a touchscreen and includes a software keyboard for entering text input, which may include stimulation parameters, patient information, etc. The display device can also include audio input and output components, such as a microphone and speaker, for example.

The system 710 further includes a stimulation probe 724 and a microelectrode assembly 722 having a microelectrode probe, which are utilized by the one or more processors 712, 714, via a driver 718, to position either probe 722, 724 into the brain of the individual for stimulation treatment. In one example configuration, the microelectrode 722 includes a wideband low-noise amplifier, which may have differential amplifying capabilities, for example: a wideband range of 0 Hz to 10 kHz, and a signal gain per channel 1 to 100,000; a data acquisition card including 1 to 12 channel high impedance analog inputs, a digital converter to USB interface, and a variable sampling frequency between 1 Hz to 50 kHz. To facilitate low-noise recording and wideband signal analysis, the size and material properties of the microelectrode probe 722 may include a tip diameter between 40-100 μm or smaller, and an impedance between 1 kOhm and 1 MOhm or lower, for example. A shielded cable (not shown) may interface with the microelectrode 722 and an analog input to the amplifier to provide shielding against stray interference from other electronic hardware and to protect low amplitude raw signals received or captured by the microelectrode probe 722.

The processor 712 is capable of processing electrophysiology data, for example, neural activity, received by the microelectrode 722 (and/or stimulation probe with signal-receiving capabilities) and recording the data into the memory 716 and/or at a remote location. The memory 716 can be tangible, non-transitory memory and can include one or several suitable memory modules, such as random access memory (RAM), read-only memory (ROM), flash memory, other types of persistent memory, etc. The memory 716 may store the training data, test data, stimulation parameters, etc., which may be configured into a data structure. The memory 716 may also store an operating system, which can be any type of suitable operating system, which can include application programming interface (API) functions that allow applications to retrieve sensor readings from the microelectrode assembly 722 and/or stimulation probe 724. For example, a software application (routine/module 720) configured to execute on the system 710 can include instructions that invoke an OS API to execute any portion of executing the machine learning prediction model, e.g., training the machine learning prediction model.

Machine learning models 726 and routines/modules 728, either of which may be in the form of a support vector machine, can be stored on the memory 716, wherein execution of any combination thereof by the processor 712 may perform at least one step in the stimulation treatment methods and machine learning techniques described above. Some example machine learning models include, and are not limited to, the training, testing, predicting, analyzing, receiving, and stimulating methods described herein. Some example routines/modules include an imaging routine for illustrating stimulation treatment data for visual inspection, MRI imaging, etc.; an interface routine for facilitating interaction between a user and the neural targeting system; a probe implanting routine to facilitate execution of the driver 718 to implant the microelectrode probe 722 and the stimulation probe 724 into the brain of an individual to be treated; etc.

It should be understood that, unless a term is expressly defined in this patent using the sentence, “As used herein, the term ‘______’ is hereby defined to mean . . . ,” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

Furthermore, although the foregoing text sets forth a detailed description of numerous different embodiments, it should be understood that the scope of the patent is defined by the words of the claims set forth at the end of this patent. The detailed description is to be construed as exemplary only and does not describe every possible embodiment because describing every possible embodiment would be impractical, if not impossible. Numerous alternative embodiments could be implemented, using either current technology or technology developed after the filing date of this patent, which would still fall within the scope of the claims. By way of example, and not limitation, the disclosure herein contemplates at least the following aspects.

Aspect 1: A method for predicting a location within the brain for stimulation treatment of an individual afflicted with a neurological illness or disorder, the method comprising: extending a microelectrode along a trajectory within the brain of the individual; receiving, via the microelectrode, electrophysiology data at a plurality of intervals along the trajectory, the electrophysiology data being indicative of neural activity of the individual; and, utilizing, via one or more processors, a machine learning model trained on clinically-determined stimulation treatment of a plurality of similarly-afflicted individuals to predict a tissue activation volume within the brain of the individual based on the received electrophysiology data of the individual.

Aspect 2: The method of aspect 1, further comprising: implanting a stimulation probe along the trajectory within the brain of the individual; and activating the stimulation probe to stimulate the predicted tissue activation volume.

Aspect 3: The method of any of aspects 1 or 2, wherein activating the stimulation probe includes one or more activation contacts and/or one or more leads.

Aspect 4: The method of any of aspects 1 through 3, wherein activating the stimulation probe to stimulate the predicted tissue activation volume includes any of the following stimulation parameters: theta, alpha, beta, high gamma, high frequency oscillations (HFO), high frequency band (HFB), theta×HFB, alpha×beta, beta×HFB, and high gamma×HFO.

Aspect 5: The method of any of aspects 1 through 4, further comprising: analyzing, via the one or more processors, the received electrophysiology data; and classifying, via the one or more processors, each interval of received electrophysiology data as being inside or outside the predicted tissue activation volume.

Aspect 6: The method of any of aspects 1 through 5, wherein a spacing between each interval along the trajectory of received electrophysiology data is 0.5 mm.

Aspect 7: The method of any of aspects 1 through 6, wherein the predicted tissue activation volume is not contiguous.

Aspect 8: A method for adjusting the stimulation treatment of an initial volume within the brain of an individual afflicted with a neurological illness or disorder, the method comprising: receiving electrophysiology data via a stimulation probe permanently implanted within the brain of the individual, the implanted stimulation probe including a stimulation parameter and configured to stimulate the initial volume within the brain; utilizing, via one or more processors, a machine learning model trained on clinically-determined stimulation treatment of a plurality of similarly-afflicted individuals to predict a tissue activation volume with the brain of the individual based on the received electrophysiology data of the individual; analyzing the predicted tissue activation volume within the brain and the stimulated initial volume within the brain; and adjusting the stimulation parameter of the stimulation probe to stimulate the predicted tissue activation volume within the brain of the individual based on the analysis of the predicted tissue activation volume and the stimulated initial volume.

Aspect 9: The method of aspect 8, wherein the stimulation parameter includes a frequency or frequency range, such as: theta, alpha, beta, high gamma, high frequency oscillations (HFO), high frequency band (HFB), theta×HFB, alpha×beta, beta×HFB, and high gamma×HFO.

Aspect 10: A method for adjusting the stimulation treatment of an initial volume within the brain of an individual afflicted with a neurological illness or disorder, the method comprising: providing a stimulation probe permanently implanted within a trajectory in the brain of the individual, the implanted stimulation probe including a stimulation parameter and configured to stimulate the initial volume within the brain; receiving electrophysiology data attained via a microelectrode probe at a plurality of intervals along the trajectory; utilizing, via one or more processors, a machine learning model trained on clinically-determined stimulation treatment of a plurality of similarly-afflicted individuals to predict a tissue activation volume with the brain of the individual based on the received electrophysiology data of the individual; analyzing, via the one or more processors, the predicted tissue activation volume within the brain and the initial volume within the brain; and adjusting, via the one or more processors, the stimulation parameter of the stimulation probe to stimulate the predicted tissue activation volume within the brain of the individual based on the analysis of the predicted tissue activation volume and the initial volume.

Aspect 11: The method of aspect 10, wherein receiving electrophysiology data includes extending a microelectrode along the trajectory within the brain of the individual.

Aspect 12: The method of any of aspects 10 or 11, wherein a spacing between each interval along the trajectory of received electrophysiology data is 0.5 mm.

Aspect 13: The method of any of aspects 10 through 12, wherein the stimulation parameter includes a frequency or frequency range, such as: theta, alpha, beta, high gamma, high frequency oscillations (HFO), high frequency band (HFB), theta×HFB, alpha×beta, beta×HFB, and high gamma×HFO.

Aspect 14 A method for predicting a location within the brain for stimulation treatment of an individual afflicted with a neurological illness or disorder, the method comprising: extending a microelectrode along a plurality of trajectories within the brain of the individual; receiving, via the microelectrode, electrophysiology data at a plurality of intervals along each of the plurality of trajectories, the electrophysiology data being indicative of neural activity of the individual; utilizing, via one or more processors, a machine learning model trained on clinically-determined stimulation treatment of a plurality of similarly-afflicted individuals to predict a plurality of tissue activation volumes within the brain of the individual based on the electrophysiology data received along the plurality of trajectories, wherein each of the predicted plurality of tissue activation volumes associated with a respective one of the plurality of trajectories; analyzing the predicted tissue activation volumes based on a stimulation treatment criteria to determine a tissue activation volume for stimulation treatment of the individual; and, selecting one of the plurality of trajectories associated with the determined tissue activation volume for treatment of the individual.

Aspect 15: The method of aspect 14, further comprising: implanting a stimulation probe along the selected trajectory within the brain of the individual; and activating the stimulation probe to stimulate the predicted tissue activation volume.

Aspect 16: The method of aspect 15, wherein activating the stimulation probe includes one or more activation contacts and/or one or more leads.

Aspect 17: The method of any of aspects 15 or 16, wherein activating the stimulation probe to stimulate the predicted tissue activation volume includes any of the following stimulation parameters: theta, alpha, beta, high gamma, high frequency oscillations (HFO), high frequency band (HFB), theta×HFB, alpha×beta, beta×HFB, and high gamma×HFO.

Aspect 18: The method of any of aspects 14 through 17, further comprising: analyzing, via the one or more processors, the received electrophysiology data; and classifying, via the one or more processors, each interval of received electrophysiology data as being inside or outside the predicted tissue activation volume.

Aspect 19: The method of any of aspects 14 through 18, wherein a spacing between each interval along the trajectory of received electrophysiology data is 0.5 mm.

Aspect 20: The method of any of aspects 14 through 19, wherein the predicted tissue activation volume is not contiguous.

Aspect 21: A system for predicting a location within the brain for stimulation treatment of an individual afflicted with a neurological illness or disorder, the system comprising: one or more processors operatively coupled to a microelectrode probe; a memory coupled to the one or more processors and including a set of instructions stored thereon, which when executed by the one or more processors cause the system to: extend the microelectrode along a trajectory within the brain of the individual; receive electrophysiology data at a plurality of intervals along the trajectory, the electrophysiology data being indicative of neural activity of the individual; and, utilize a machine learning model trained on clinically-determined stimulation treatment of a plurality of similarly-afflicted individuals to predict a tissue activation volume within the brain of the individual based on the received electrophysiology data of the individual.

What is claimed is:

1. A method for predicting a location within the brain for stimulation treatment of an individual afflicted with a neurological illness or disorder, the method comprising:
extending a microelectrode along a trajectory within the brain of the individual;
receiving, via the microelectrode, electrophysiology data at a plurality of intervals along the trajectory, the electrophysiology data being indicative of neural activity of the individual; and,
utilizing, via one or more processors, a machine learning model trained on clinically-determined stimulation treatment of a plurality of similarly-afflicted individuals to predict a tissue activation volume within the brain of the individual based on the received electrophysiology data of the individual.

2. The method of claim 1, further comprising:
implanting a stimulation probe along the trajectory within the brain of the individual; and
activating the stimulation probe to stimulate the predicted tissue activation volume.

3. The method of claim 2, wherein activating the stimulation probe includes one or more activation contacts and/or one or more leads.

4. The method of claim 2, wherein activating the stimulation probe to stimulate the predicted tissue activation volume includes any of the following stimulation parameters: theta, alpha, beta, high gamma, high frequency oscillations (HFO), high frequency band (HFB), theta×HFB, alpha×beta, beta×HFB, and high gamma×HFO.

5. The method of claim 1, further comprising:
analyzing, via the one or more processors, the received electrophysiology data; and
classifying, via the one or more processors, each interval of received electrophysiology data as being inside or outside the predicted tissue activation volume.

6. The method of claim 1, wherein a spacing between each interval along the trajectory of received electrophysiology data is 0.5 mm.

7. The method of claim 1, wherein the predicted tissue activation volume is not contiguous.

8. A method for adjusting the stimulation treatment of an initial volume within the brain of an individual afflicted with a neurological illness or disorder, the method comprising:
receiving electrophysiology data via a stimulation probe permanently implanted within the brain of the individual, the implanted stimulation probe including a stimulation parameter and configured to stimulate the initial volume within the brain;
utilizing, via one or more processors, a machine learning model trained on clinically-determined stimulation treatment of a plurality of similarly-afflicted individuals to predict a tissue activation volume with the brain of the individual based on the received electrophysiology data of the individual;
analyzing the predicted tissue activation volume within the brain and the stimulated initial volume within the brain; and
adjusting the stimulation parameter of the stimulation probe to stimulate the predicted tissue activation volume within the brain of the individual based on the analysis of the predicted tissue activation volume and the stimulated initial volume.

9. The method of claim 8, wherein the stimulation parameter includes a frequency or frequency range, such as: theta, alpha, beta, high gamma, high frequency oscillations (HFO), high frequency band (HFB), theta×HFB, alpha×beta, beta×HFB, and high gamma×HFO.

10. A method for adjusting the stimulation treatment of an initial volume within the brain of an individual afflicted with a neurological illness or disorder, the method comprising:
providing a stimulation probe permanently implanted within a trajectory in the brain of the individual, the implanted stimulation probe including a stimulation parameter and configured to stimulate the initial volume within the brain;
receiving electrophysiology data attained via a microelectrode probe at a plurality of intervals along the trajectory;
utilizing, via one or more processors, a machine learning model trained on clinically-determined stimulation treatment of a plurality of similarly-afflicted individuals to predict a tissue activation volume with the brain of the individual based on the received electrophysiology data of the individual;
analyzing, via the one or more processors, the predicted tissue activation volume within the brain and the initial volume within the brain; and
adjusting, via the one or more processors, the stimulation parameter of the stimulation probe to stimulate the predicted tissue activation volume within the brain of the individual based on the analysis of the predicted tissue activation volume and the initial volume.

11. The method of claim 10, wherein receiving electrophysiology data includes extending a microelectrode along the trajectory within the brain of the individual.

12. The method of claim 10, wherein a spacing between each interval along the trajectory of received electrophysiology data is 0.5 mm.

13. The method of claim 10, wherein the stimulation parameter includes a frequency or frequency range, such as: theta, alpha, beta, high gamma, high frequency oscillations (HFO), high frequency band (HFB), theta×HFB, alpha×beta, beta×HFB, and high gamma×HFO.

14. A method for predicting a location within the brain for stimulation treatment of an individual afflicted with a neurological illness or disorder, the method comprising:
extending a microelectrode along a plurality of trajectories within the brain of the individual;
receiving, via the microelectrode, electrophysiology data at a plurality of intervals along each of the plurality of trajectories, the electrophysiology data being indicative of neural activity of the individual;
utilizing, via one or more processors, a machine learning model trained on clinically-determined stimulation treatment of a plurality of similarly-afflicted individuals to predict a plurality of tissue activation volumes within the brain of the individual based on the electrophysiology data received along the plurality of trajectories, wherein each of the predicted plurality of tissue activation volumes associated with a respective one of the plurality of trajectories;
analyzing the predicted tissue activation volumes based on a stimulation treatment criteria to determine a tissue activation volume for stimulation treatment of the individual; and, selecting one of the plurality of trajectories associated with the determined tissue activation volume for treatment of the individual.

15. The method of claim 14, further comprising:
implanting a stimulation probe along the selected trajectory within the brain of the individual; and
activating the stimulation probe to stimulate the predicted tissue activation volume.

16. The method of claim 15, wherein activating the stimulation probe includes one or more activation contacts and/or one or more leads.

17. The method of claim 15, wherein activating the stimulation probe to stimulate the predicted tissue activation volume includes any of the following stimulation parameters: theta, alpha, beta, high gamma, high frequency oscillations (HFO), high frequency band (HFB), theta×HFB, alpha×beta, beta×HFB, and high gamma×HFO.

18. The method of claim 14, further comprising:
analyzing, via the one or more processors, the received electrophysiology data; and
classifying, via the one or more processors, each interval of received electrophysiology data as being inside or outside the predicted tissue activation volume.

19. The method of claim 14, wherein a spacing between each interval along the trajectory of received electrophysiology data is 0.5 mm.

20. A system for predicting a location within the brain for stimulation treatment of an individual afflicted with a neurological illness or disorder, the system comprising:
one or more processors operatively coupled to a microelectrode probe;
a memory coupled to the one or more processors and including a set of instructions stored thereon, which when executed by the one or more processors cause the system to:
extend the microelectrode along a trajectory within the brain of the individual;
receive electrophysiology data at a plurality of intervals along the trajectory, the electrophysiology data being indicative of neural activity of the individual; and,
utilize a machine learning model trained on clinically-determined stimulation treatment of a plurality of similarly-afflicted individuals to predict a tissue activation volume within the brain of the individual based on the received electrophysiology data of the individual.
